(19) United States
(12) Patent Application Publication
(10) Pub. No.: US 2022/0104546 A1
(43) Pub. Date: Apr. 7, 2022

(54) VAPOR DISPENSING APPARATUS HAVING A TUBULAR WICK SECTION WITH AN INTERNAL HEATING ELEMENT AND REFILLS THEREFOR

(71) Applicant: HENKEL IP & HOLDING GmbH, Dusseldorf (DE)

(72) Inventors: Mark A. Granja, Danbury, CT (US); Saumya Amar Singh, Charlotte, NC (US); Jamie Scott Anderson, Sandy Hook, CT (US)

(73) Assignee: HENKEL IP & HOLDING GMBH, Duesseldorf (DE)

(21) Appl. No.: 17/491,339

(22) Filed: Sep. 30, 2021

Related U.S. Application Data
(60) Provisional application No. 63/087,682, filed on Oct. 5, 2020.

(51) Int. Cl. A24F 40/42; A24F 40/46; A24F 40/44

(52) U.S. Cl. CPC A24F 40/42; A24F 40/44; A24F 40/46

(57) ABSTRACT

A vapor dispensing apparatus includes a housing configured to support a refill therein. The housing has a vent formed therein. The refill includes a bottle having a body defining an interior volume for storing a vaporizable liquid. A wick, configured to draw the vaporizable liquid out of the bottle, includes an inferior portion disposed in the interior volume and a superior portion extending from the inferior portion and terminating outside the bottle adjacent the vent. The wick is an elongated member having a hollow wick core formed in the superior portion to provide a tubular wick section. An electric warmer is supported in the housing and configured to couple with a power supply. The electric warmer includes a heating element received in the hollow wick core for warming the wick above an ambient temperature.

### CROSS-REFERENCE TO RELATED APPLICATIONS

This application claims the benefit of U.S. Provisional Application No. 63/087,682, which was filed on Oct. 5, 2020. The entire disclosure of this provisional application is incorporated herein by reference.

### FIELD OF THE INVENTION

The following disclosure relates generally to liquid vaporizers for dispensing volatile liquid materials such as fragrance oils, deodorants, insecticides and the like, and, more particularly, to a vapor dispensing apparatus including an elongated wick having a tubular wick section with an internal heating element and refills therefor.

### BACKGROUND OF THE INVENTION

Electrical liquid vaporizers (also referred to as scented warmers) are generally known in the art. Such electrical liquid vaporizers include a housing unit configured to receive a replaceable bottle or simply a refill. The refill includes a bottle having a wick passing therethrough which the volatile liquid migrates to a portion of the wick extending from the bottle and exposed to the environment. The exposed portion of the wick is generally heated by a heating element disposed within the housing unit proximate the wick for facilitating the vaporization of the volatile liquid into the environment.

Conventionally, these vaporizers utilize a solid core wick. The heating element uses a single resistor supported in the housing adjacent an outer surface of the wick or, alternately, a pair of resistors disposed in a ring-shaped configuration supported around the outer surface of the wick as a heat source to promote volatilization of liquid materials in the wick. Factors for enhancing volatilization of liquid materials in the wick include the amount of wick in proximity to the heating elements, as well as the overall surface area of the wick for evaporating the volatile liquid material.

There is a continuing effort in this art to improve the efficiency and/or fragrance intensity emitted from electrical liquid vaporizers of this type. With these conventional vaporizers this is principally achieved by adjusting the chemistry of the volatile liquid material to evaporate more quickly or by varying the resistance of the heating element thereby increasing the temperature of the wick. Accordingly, there is a need in this art to find additional means to improve the efficiency and/or fragrance intensity emitted for such vaporizers.

### BRIEF SUMMARY OF THE INVENTION

Embodiments for vapor dispensing apparatuses (or simply vaporizers) and replaceable bottles (or simply refills) containing the volatile liquid material are provided with increased surface area of the wick without modifying the overall size of the vaporizer and improving the heat transfer from the heating element to the wick. In particular, the vaporizers and refills described herein utilize a wick including an elongated member having a hollow wick core extending at least partially through the elongated member to form a tubular wick section for increasing the available surface area from which the volatile liquid material is evaporated. An electric warmer is supported in a housing and configured to electrically couple with a power source. The electric warmer includes an elongated heating element disposed in the hollow wick core to warm an inner surface of the wick for enhancing volatilization of liquid materials in the wick. A complimentary configuration of the elongated heating element interlocks with the hollow wick core to provide a lock and key feature for excluding the use of other, third party refills that do not have this feature.

In an embodiment, a vapor dispensing apparatus includes a housing and a replaceable bottle or refill. The housing has a vent and an electric warmer supported therein and configured to electrically couple with a power source. The refill is supported in the housing and has a body defining an interior volume for storing a vaporizable liquid and a wick configured to draw the vaporizable liquid out of the bottle. The wick is an elongated member having an inferior portion disposed in the interior volume and a superior portion extending from the inferior portion and terminating at a second end adjacent the vent. A hollow wick core extends through the superior portion to form a tubular wick section having an inner surface. The electric warmer includes an elongated heating element received in the hollow wick core for warming the wick above an ambient temperature.

In another embodiment, a refill for a vapor dispensing apparatus includes a bottle configured to be supported in a housing and having a body defining an interior volume for storing a vaporizable liquid. The refill also includes a wick configured to draw the vaporizable liquid out of the bottle. The wick is an elongated member having an inferior portion disposed in the interior volume and a superior portion extending from the inferior portion and terminating outside the body. A hollow wick core extends through the superior portion to form a tubular wick section having an inner surface. The hollow wick core is configured to receive an elongated heating element therein for warming the wick above an ambient temperature.

In another embodiment, the vapor dispensing apparatus includes a housing and a refill having a bottle and a wick. The housing has a first coupling element, a vent and an electric warmer supported therein and configured to couple with a power source. The bottle includes a body defining an interior volume for storing a vaporizable liquid and a finish extending from the body. The finish has a second coupling element formed thereon which cooperates with the first coupling element to releasably secure the bottle to the housing. The wick is disposed through the finish in the bottle for drawing the vaporizable liquid therefrom. The wick is an elongated member having an inferior portion disposed in the interior volume and a superior portion extending from the inferior portion and terminating outside the body at an end adjacent the vent. A hollow wick core extends through the superior portion to form a tubular wick section having an inner surface. The inferior portion and superior portion together define an outer surface. The electric warmer includes a rod-shaped heating element received in the hollow wick core. The rod-shaped heating element has an elongated heating surface for radially heating the inner surface of the wick to warm the wick above an ambient temperature.

The foregoing statements are provided by way of non-limiting examples only. Various additional examples, aspects, and other features of embodiments of the present disclosure are encompassed by the present disclosure including the detailed description and claims set out below.

### BRIEF DESCRIPTION OF THE DRAWINGS

Further details of the vapor dispensing apparatus having an interlocked heated core and refills therefor will hereinafter be described in conjunction with the following figures, wherein like numerals denote like elements, and:

FIG. 1 is an expanded schematic representation of a vapor dispensing apparatus in accordance with the present disclosure;

FIG. 2 is an assembled schematic representation of the vapor dispensing apparatus shown in FIG. 1;

FIG. 3 illustrates the refill and electric warmer for the vapor dispensing apparatus;

FIG. 4 is a detail showing the wick with a blind bore forming the hollow wick core;

FIG. 5 is a detail showing the wick with a through bore and a bung forming the hollow wick core; and

FIG. 6 is a cross-section taken through the wick and elongated heating element at line VI-VI shown in FIG. 3.

For simplicity and clarity of illustration, descriptions and details of well-known features and techniques may be omitted to avoid unnecessarily obscuring the exemplary and non-limiting embodiments of the present disclosure described in the subsequent Detailed Description. It should further be understood that features or elements appearing in the accompanying figures are not necessarily drawn to scale unless otherwise stated.

### DETAILED DESCRIPTION OF THE INVENTION

The following Detailed Description is merely exemplary in nature and is not intended to limit the present disclosure or the application and uses of the same. The term “exemplary,” as appearing throughout this document, is synonymous with the term “example” and is utilized repeatedly below to emphasize that the following description provides multiple non-limiting examples of the present disclosure and should not be construed to restrict or otherwise limit the scope of the claimed subject matter in any respect.

The present disclosure describes a vapor dispensing apparatus having a tubular wick with increased surface area in proximity to the heating element of the electric warmer for increasing the temperature of the wick. As a result, the intensity of the fragrance emitted from the vapor dispensing apparatus is improved by increasing the surface area of the wick without modifying the overall size of the vaporizer and improving the heat transfer from the heating element to the wick. In particular, the wick includes an elongated member having a hollow wick core which provides inner and outer surfaces for evaporating the volatile liquid material. An electric warmer has an elongated heating element disposed in the hollow wick core so that the inner surface is exposed to heat generated by the heating element. In other words, the heating element radiates heat outwardly though the inner surface to the outer surface of the tubular wick.

In one embodiment, the vapor dispensing apparatus includes a lock and key mechanism, wherein the hollow wick core provides a lock feature and the electric warmer and more particularly a complimentary cross-section of the elongated heating element to the hollow wick core provides a key feature. For example, the size and shape of the hollow wick core matches the size and shape of the elongated heating element. In this way, these features are complimentary to each other for making the wick compatible with the housing, and thus providing a lock and key mechanism which requires the correct wick or refill to be used with the vapor dispensing apparatus.

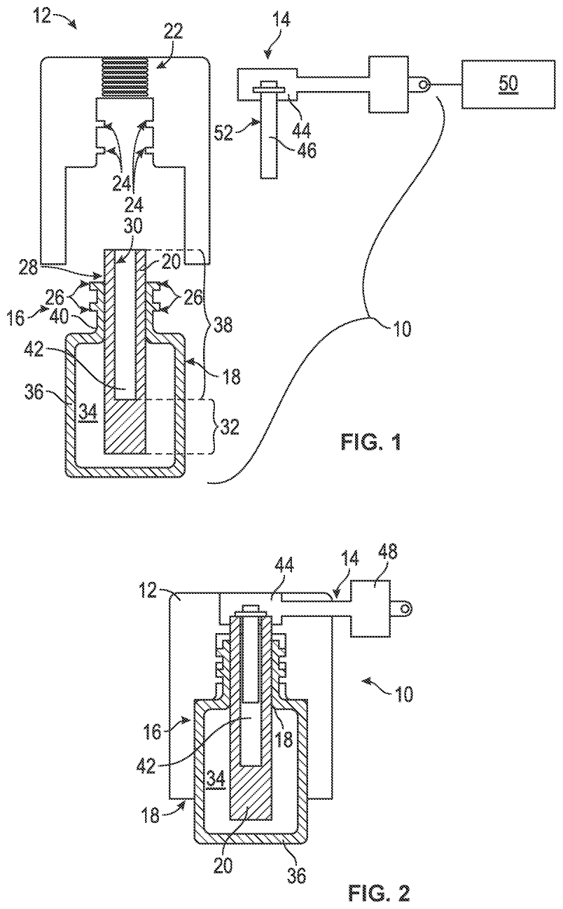

With reference now to FIGS. 1 and 2, a vapor dispensing apparatus 10 is illustrated in expanded and assembled form, respectively. The vapor dispensing apparatus 10 includes a housing 12, an electric warmer 14 and a replaceable bottle or simply a refill 16. The refill 16 includes a bottle 18 and a wick 20 extending out of the bottle 18. These components of the vapor dispensing apparatus 10 are illustrated as distinct elements each suitably adapted to engage with the others to form a functional vapor dispensing apparatus 10. With that said, one skilled in the art will appreciate that two or more of these components may be integrated together. For example, the housing 12 and the electric warmer 14 may be assembled in a unitary configuration that is adapted to receive a refill 16 for providing a refillable vapor dispensing apparatus. In another example, the housing 12, the electric warmer 14 and the refill 16 may be assembled in a unitary configuration to provide a disposable vapor dispensing apparatus 10. Thus, these and other alternative configurations are intended to fall within the scope of the disclosure provided herein.

The housing 12 has a vent 22 and a first coupling element 24 formed therein. The refill 16 has a second coupling element 26 which cooperates with the first coupling element 24 to position and releasably secure the refill 16 in the housing 12. The first and second coupling elements 24, 26 may be configured to provide a “snap-and-fit” coupling for releasably securing the refill 16 into the housing 12. One skilled in the art should appreciate that the coupling elements 24, 26 are schematically illustrated in the drawings and that other coupling mechanism—such as a bayonet coupling, a threaded coupling or other suitable coupling mechanism—now known or hereafter devised may also be used. When the refill 16 is secured to the housing 12, the wick 20 is disposed adjacent the vent 22 so that vapors evaporated from the wick 20 are emitted out of the vent 22 into the surrounding environment.

The wick 20 is an elongated member having an inferior portion 32 and a superior portion 38. The inferior portion 32 of the wick 20 is disposed in an interior volume 34 formed by body 36 of the bottle 18. The superior portion 38 extends from the inferior portion 32 and terminates beyond a neck or finish 40 of the bottle 18. A hollow wick core 42 extends through the superior portion 38 to form a tubular wick section having an inner surface 30. The inferior portion and superior portion together define and an outer surface 28. As used herein, the inferior portion 32 represents a length L₁ of the wick 20, which forms a solid wick section, and the superior portion 38 of the wick 20 represents a length L₂ of the wick 20 having the hollow wick core 42, which forms the tubular wick section. While the figures illustrate the superior portion 38 extending through the finish 40 of the bottle, one skilled in the art should understand that either the inferior portion 32 or the superior portion 38 may extend through the finish 40 of the bottle 18. Likewise, the wick 20 is illustrated in the figures as a right cylinder having a circular cross-section; however, the term “elongated” is used more broadly herein to encompass various wick configurations, which are relatively long when compared with the transverse dimensions and may have a circular, oval or closed polygonal cross-sectional configuration.

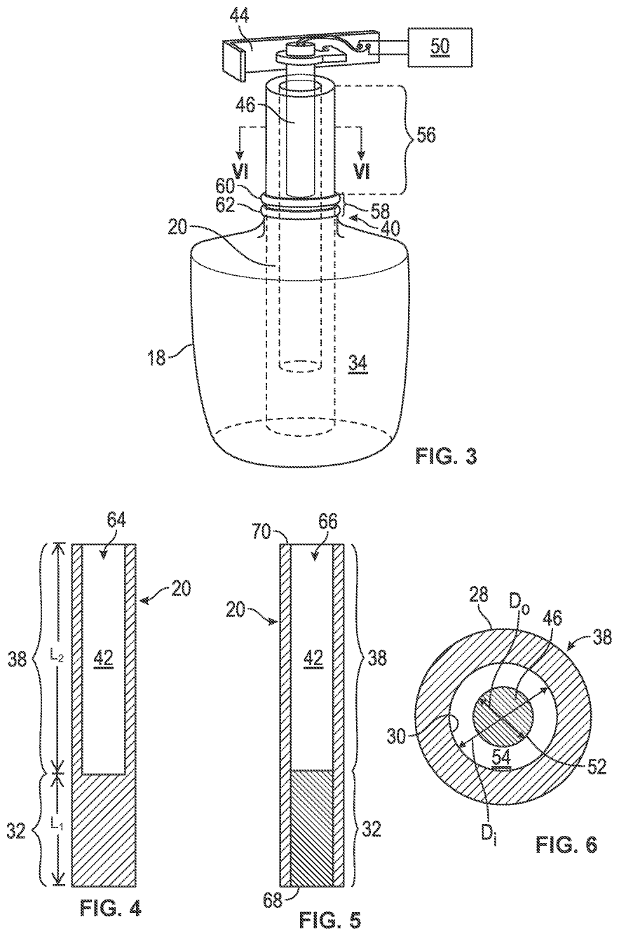

The electric warmer 14 is also supported in the housing 12 and functions to warm the wick 20 above an ambient temperature of the environment. In particular, the electric warmer 14 includes a substrate 44, such as a printed circuit board, that is mounted to the housing 12. An elongated heating element 46 is suspended from the substrate 44. The electric warmer 14 also includes a plug 48 for electrically coupling the heating element 46 to a power source 50. The electric warmer 14 may include one or more electric circuits, for example disposed on the substrate 44 for switching the heating element 46 ON and OFF and/or for regulating the power supplied from the power source 50 to the heating element 46. Additional details concerning these electric circuits are provided in U.S. Pat. No. 6,661,967 entitled Variable Temperature Vaporizer, the disclosure of which is expressly incorporated by reference herein. When the refill 16 is secured to the housing 12, the elongated heating element 46 is positioned within the hollow wick core 42. While the heating element 46 is shown as partially extending into the hollow wick core 42, one skilled in the art should understand that the heating element 42 may extend completely into the hollow wick core 42 for warming the entire length of the tubular wick section. The elongated heating element 46 has a heating surface 52 for radially heating the inner surface 30 to warm the wick 20 above the ambient temperature. As illustrated in FIG. 3, the elongated heating element 46 is a cylindrical heating element, which is complimentary with the hollow wick core 42. However, the term “elongated heating element” as used herein is not so limited and is intended to include any shape, which is relatively long when compared with the transverse dimensions of the heating element 46 and may have a circular, oval or closed polygonal cross-sectional configuration. As best seen in FIGS. 3 and 6, an air gap 54 is formed between the heating surface 52 of the heating element 46 and the inner surface 30 of the wick 20. FIG. 3 illustrates an exemplary embodiment of the relative positioning and placement of the wick 20 and the heating element 46, as well as a heated region 56 of the wick 20. Volatile liquid material is drawn into the wick 20 where it is heated and evaporates from the outer and inner surfaces 28, 30 of the wick 20 and passes through vent 22 into the environment.

Refill 16 is configured to hold a vaporizable liquid material in the interior volume 34. In accordance with various aspects of the present invention, the vaporizable liquid material can be any number of conventional materials evaporated from a vaporizer including fragrances, disinfectants, sanitizing agents, insect repellants, insecticides and the like. In accordance with one aspect of the present invention, the material to be volatilized comprises a fragrance material and vapor dispensing apparatus 10 is used as an air freshening device. In this manner, refill 16 is filled with a fragrance liquid and is inserted into housing 12 such that the fragrance liquid can be vaporized through operation of the electric warmer 14 which promotes or encourages vaporization of the fragrance from the wick 20.

Refill 16 is suitably sized for use in connection with household use. In accordance with various aspects of the present invention, bottle 18 is configured to hold between about 25 milliliters and about 75 milliliters of liquid material, and preferably between about 35 milliliters and about 50 milliliters of liquid. In the presently described, non-limiting embodiment, the weight and moment of the apparatus 10, inclusive of a full refill 16, is such that the center of gravity is appropriately positioned, and the weight is less than that which would otherwise cause the apparatus 10 to be unstable when plugged into the power source 50.

Bottle 18 may be a conventional bottle or similar device configured to receive a volatile liquid material while holding the wick 20 firmly in place. Generally, wick 20 will be secured to bottle 18 by a wick securement system 58. For example, wick 20 may be secured within a wick retainer 60, which in turn is attached to an attachment ring 62 that is crimped or otherwise attached to the finish 40 of bottle 18. In an embodiment, bottle 18 is a plastic bottle compatible with the material to be vaporized. For example, bottle 18 may be formed of polypropylene and/or PET. Housing 12 may also be a plastic structure fabricated from a suitable material, such as polypropylene, a high-density polyethylene or a polystyrene. However, in certain applications, it may be desirable for bottle 18 to be formed of other materials such as glass or the like. In accordance with various aspects of the present invention, wick securement system 58 may be formed using plastic, metal or other suitable materials. It should be appreciated that the particular composition of housing 12, bottle 18, and/or securement system 58 may be modified to any suitable material composition as is now known or hereafter devised by those skilled in the art.

The wick 20 may be formed from any conventional wick material. Suitable wick materials include porous/sintered plastics such as high-density polyethylene and polypropylene, polyester, bonded fibers, glass sintered fibers, ceramic materials, carbon fibers, sintered carbon, wood, compressed wood composites bundled or woven material fibers, bundled or manmade fibers. In an embodiment for a fragrance vaporizer, a polyester wick having a relatively high melting point is effective for wicking fragrance from the interior volume 34 into the wick 20 and evaporating the fragrance from the wick 20 into the environment. However, wick 20 can be formed of any suitable material now known or hereafter devised by those skilled in the art. The wick 20 may be fabricated using any conventional process, such as molding or extruding and drilling.

As explained above, the wick 20 is an elongated member having a hollow wick core 42. The inferior portion 32 is configured to extend into the volatile liquid material in the interior volume 34 and the superior portion 38 extends out of the bottle 18. As such, volatile liquid material could potentially leak out for the bottle 18 through the wick 20 and out the hollow wick core 42 when the refill 16 is tipped over or the apparatus 10 is plugged in upside down. To negate this concern, the wick 20, and specifically the hollow wick core 42 is configured to resist such liquid flow. For example, as illustrated in FIG. 4, the hollow wick core 42 may be formed as a blind bore 64 only extending through the superior portion 38 of the wick 20. Alternately, as illustrated in FIG. 5, a through bore 66 may be formed through the wick 20 extending from a first end 68 on the inferior portion 32 to a second end 70 on the superior portion 38. A bung 72 is disposed in the through bore 66 at the first end 68 to make the inferior portion 32 a solid core wick section so that the hollow wick core 42 extend through only the superior portion 38 of the wick 20. Bung 72 may be made using a similar material as the wick 20 or alternately using a diverse material.

As best illustrated in FIGS. 3 and 6, the hollow wick core 42 and the elongated heating element are configured as right cylinders having circular cross-sections. In this way, the hollow wick core 42 provides a lock feature and the heating element 46 provides a key feature that is complimentary with the lock feature and make the refill 16 exclusively compatible with the housing 12. For example, the outer diameter Dₒ of the heating element 46 is less than the inner diameter Dᵢ of the hollow wick core 42 so that the heating element 46 can be received therein. One skilled in the art will appreciate that the lock feature and key feature may include other configurations and/or cross-sections which are complimentary in nature for making the refill 16 exclusively compatible with the housing 12.

The electric warmer 14, and more specifically the heating element 46 operates to warm the wick 20 above the ambient temperature. In embodiments where the vapor dispensing apparatus 10 is configured as a fragrance dispenser and the volatile liquid material is a fragrance liquid, the heating element 46 should function to heat the wick 20 to an operating temperature compatible with the wick material and the formulation of the volatile liquid material. To affect this range of heating, the heating element 46 may be configured as a resistive heating element such as a single resistor, a variable resistor, sets of multiple resistors in parallel and/or series arrangements as well as other resistive means well known in the art. Alternately, the heating element 46 may be configured as a positive temperature coefficient (PTC) thermistor, which suitably adjusts the resistance to compensate for variations of electrical voltage and/or current in a specified range. Within this range, the energy output of the vaporizer and the wick temperature can generally be maintained with respect to the corresponding voltage/current flux.

Terms such as “first” and “second” have been utilized above to describe similar features or characteristics (e.g., rotational directions) in view of the order of introduction during the course of description. In other sections of this Application, such terms can be varied, as appropriate, to reflect a different order of introduction. While at least one exemplary embodiment has been presented in the foregoing Detailed Description, it should be appreciated that a vast number of variations exist. It should also be appreciated that the exemplary embodiment or exemplary embodiments are only examples, and are not intended to limit the scope, applicability, or configuration of the invention in any way. Rather, the foregoing Detailed Description will provide those skilled in the art with a convenient road map for implementing an exemplary embodiment of the invention. It is understood that various changes may be made in the function and arrangement of elements described in an exemplary embodiment without departing from the scope of the invention as set forth in the appended claims.

What is claimed is:

1. A vapor dispensing apparatus comprising:
a housing having a vent and an electric warmer supported therein, wherein the electric warmer is configured to electrically couple with a power source; and
a bottle supported in the housing and having a body defining an interior volume for storing a vaporizable liquid, and a wick configured to draw the vaporizable liquid out of the bottle, the wick having an inferior portion disposed in the interior volume and a superior portion extending from the inferior portion and terminating adjacent the vent, wherein the wick comprises an elongated member having a hollow wick core formed in the superior portion; and
wherein the electric warmer includes a heating element received in the hollow wick core for warming the wick above an ambient temperature.

2. The vapor dispensing apparatus according to claim 1, wherein the inferior portion has a solid core wick section, and the superior portion has a tubular wick section.

3. The vapor dispensing apparatus according to claim 1, wherein the heating element comprises an elongated heating element.

4. The vapor dispensing apparatus according to claim 3, wherein the elongated heating element comprises a rod-shaped heating element having a cylindrical heating surface for radially heating the inner surface of the wick.

5. The vapor dispensing apparatus according to claim 3, wherein an air gap is formed between the elongated heating element and the inner surface of the superior portion.

6. The vapor dispensing apparatus according to claim 1, wherein a shape of the heating element is complimentary to a shape of the hollow wick core.

7. The vapor dispensing apparatus according to claim 1, wherein the hollow wick core comprises a lock feature and the heating element comprises a key feature, wherein the key feature is complimentary with the lock feature for making the refill compatible with the housing.

8. The vapor dispensing apparatus according to claim 7, wherein the lock features is a cross-section of the hollow wick core and the key feature is a cross-section of the heating element.

9. The vapor dispensing apparatus according to claim 1, wherein the elongated member has a blind bore formed therein to define the superior portion of the wick.

10. The vapor dispensing apparatus according to claim 1, wherein the elongated member includes a through bore extending therethrough, the wick further comprising a bung disposed in the through bore at an end of the inferior portion.

11. The vapor dispensing apparatus according to claim 1 wherein the heating element is a resistive heating element.

12. The vapor dispensing apparatus according to claim 1 wherein the heating element is a positive temperature coefficient (PTC) heating element.

13. A refill for a vapor dispensing apparatus having a housing supporting an electric warmer with an elongated heating element electrically couple with a power source, the refill comprising:
a bottle configured to be supported in the housing and having a body defining an interior volume for storing a vaporizable liquid; and
a wick configured to draw the vaporizable liquid out of the bottle, the wick having an inferior portion disposed in the interior volume and a superior portion extending from the inferior portion and terminating outside the body, wherein the wick comprises an elongated member having a hollow wick core formed in the superior portion and configured to receive the elongated heating element therein.

14. The refill according to claim 13, wherein the inferior portion has a solid core wick section, and the superior portion has a tubular wick section.

15. The refill according to claim 13, wherein a shape of the hollow wick core is configured to be compatible with a shape of the elongated heating element.

16. The refill according to claim 15, wherein the hollow wick core comprises a lock feature which is configured to be complimentary to a key feature on the elongated heating element for making the refill compatible with the housing.

17. The refill according to claim 16, wherein the lock features is a cross-section of the hollow wick core.

18. The refill according to claim 13, wherein the elongated member includes a blind bore formed therein defining the superior portion.

19. The refill to claim 13, wherein the elongated member includes a through bore extending therethrough, the wick further comprising a bung disposed at an end of the inferior portion in the through bore to plug the through bore.

20. A vapor dispensing apparatus comprising:
a housing having a first coupling element, a vent and an electric warmer supported therein, wherein the electric warmer is configured to electrically couple with a power source; and
a bottle having a body defining an interior volume for storing a vaporizable liquid and a finish extending from the body, the finish having a second coupling element formed thereon which cooperates with the first coupling element to releasably secure the bottle to the housing; and
a wick disposed through the finish in the bottle for drawing the vaporizable liquid therefrom, the wick having an outer surface, an inferior portion with a solid core wick section disposed in the interior volume and a superior portion extending from the inferior portion and terminating outside the body adjacent the vent, wherein a hollow wick core extends through the superior portion to form a tubular wick section having an inner surface;
wherein the electric warmer includes a rod-shaped heating element received in the hollow wick core and having a cylindrical heating surface for radially heating the inner surface area of the wick to warm the wick above an ambient temperature.
